(19) United States
(12) Patent Application Publication
(10) Pub. No.: US 2025/0045843 A1
(43) Pub. Date: Feb. 6, 2025

(54) FRAGRANCE RECIPE/COMPOSITION FROM TARGET TEMPORAL ODOUR PROFILES

(71) Applicant: BASF SE, Ludwigshafen am Rhein (DE)

(72) Inventors: Oliver SALA, Ludwigshafen am Rhein (DE); Rebecca SURE, Ludwigshafen am Rhein (DE); Miriam MATHEA, Ludwigshafen am Rhein (DE)

(21) Appl. No.: 18/718,463

(22) PCT Filed: Dec. 16, 2022
(86) PCT No.: PCT/EP2022/086429

§ 371 Date: Jun. 11, 2024

(30) Foreign Application Priority Data
Dec. 17, 2021 (EP) 21215500.6

(51) Int. Cl. G06Q 50/04; G16C 20/50

(52) U.S. Cl. CPC G06Q 50/04; G16C 20/50

(57) ABSTRACT

In order to provide a more reliable process for target temporal odour producing fragrance products, a computer-implemented method (100) is provided for generating a recipe profile of a fragrance product having a target temporal odour profile, wherein the fragrance product comprises a fragrance composition with one or a plurality of fragrance ingredient(s), the computer-implemente d method comprising the steps of: a) providing (110) the target temporal odour profile, wherein the target temporal odour profile comprises a time-dependent fractural amount of a plurality of odour families over a predetermined period of time, which is indicative of a desired evaporation behaviour of each odour family in the fragrance composition; b) providing (120), for each odour family, one or more fragrance ingredients having olfactive contributions matching the respective odour family; c) selecting (130) at least one ingredient from each odour family to form one or more recipes of the fragrance product, wherein each recipe comprises fragrance composition data associated with the fragrance ingredients of the recipe; d) determining (140) a temporal odour profile for each recipe; e) determining (150) a distance of each determined temporal odour profile of the one or more recipes to the target temporal odour profile of the fragrance product; f) selecting (160) at least one recipe from the one or more recipes that has a distance satisfying a predefined criterion; and g) providing (170) a recipe profile of the at least one selected recipe preferably usable for production of the fragrance product.

### FIELD OF THE INVENTION

The present invention relates to a computer-implemented method and an apparatus for generating a recipe profile of a fragrance product having a target temporal odour profile, to a method and an apparatus for monitoring production of a fragrance product, to a method and an apparatus for validating production of a fragrance product, to a computer program element, and to use of recipe file.

### TECHNICAL BACKGROUND

State of the art fragrance compositions are prepared from a palette of different fragrance ingredients which are known for their different olfactive characters. When creating a fragrance the perfumer selects ingredients and combines them based on their olfactive character and their relative proportions. This process is usually guided by the experience of the perfumer which allows her or him to form a reasonable mental impression of the odour of the composition.

### SUMMARY OF THE INVENTION

There may be a need to improve the generation of a recipe profile for a fragrance product in order to provide a more reliable process for producing fragrance products.

According to a first aspect of the present invention, there is provided a computer-implemented method (100) for generating a recipe profile of a fragrance product having a target temporal odour profile, wherein the fragrance product comprises a fragrance composition with one or a plurality of fragrance ingredient(s) and a matrix, comprising the steps of:
a) providing (110) the target temporal odour profile, wherein the target temporal odour profile comprises a time-dependent fractural amount of a plurality of odour families over a predetermined period of time, which is indicative of a desired evaporation behaviour of each odour family in the fragrance composition;
b) providing (120), for each odour family, one or more fragrance ingredients having olfactive contributions matching the respective odour family;
c) selecting (130) at least one ingredient from each odour family to form one or more recipes of the fragrance product, wherein each recipe comprises fragrance composition data associated with the fragrance ingredients of the recipe;
d) determining (140) a temporal odour profile for each recipe;
e) determining (150) a distance of each determined temporal odour profile of the one or more recipes to the target temporal odour profile of the fragrance product;
f) selecting (160) at least one recipe from the one or more recipes that has a distance satisfying a predefined criterion; and
g) providing (170) a recipe profile of the at least one selected recipe preferably usable for production of the fragrance product.

In other words, a computer-implemented method is proposed to determine a fragrance composition from a target temporal odour profile. The target temporal odour profile may comprise a time-dependent fractural amount of a plurality of odour families in a gas phase and/or condense phase over a predetermined period of time. The target temporal odour profile may comprise a plurality of evaporation regimes over the predetermined period of time. Each evaporation regime corresponds to a time segment in the predetermined period of time. Different time segments have different evaporation profile and are therefore referred to as evaporation regimes. The fractural amount of each odour family may be represented by an area in the evaporation regimes, which may also be referred to as olfactory area. An example of the target temporal odour profile is shown in FIG. 4 and will be discussed hereinafter.

From several fragrance ingredients descriptions of the odour families and their contribution to the total olfactive perceptions is already known and can be retrieved from databases. Such databases can be commercially available databases such as e.g. ScenTree, Flavornet, GoodScents, SuperScent or Sigma-Aldrich or an internal database. Such a database usually lists the name and the structure of the fragrance ingredient together with several descriptions of the main and sub-odour families and optional additional attributes of the fragrance ingredient, such as e.g. molecular weight or boiling points, if available.

For each odour family, a list of fragrance ingredients may be retrieved from the database based on the descriptions. Then, one or more recipes may be proposed by selecting fragrance ingredients from the retrieved fragrance ingredients. All fragrance ingredients in one recipe may be defined by listing them and defining their amounts in the recipe.

A temporal odour profile of the one or more recipes is then determined. This will be explained hereinafter and in particular with respect to the example shown in FIG. 5.

A distance of each of the determined temporal odour profiles of the one or more recipes to the target temporal odour profile is determined. For example, the determined temporal odour profile may also be divided into a plurality of evaporation regimes similar to that of the target temporal regimes. The fractural amounts of the odour families in each evaporation regime of the determined temporal odour profile may be compared with that of the target temporal odour profile to determine the distance.

Then, at least one recipe is selected from the one or more recipes that has a distance satisfying a predefined criterion. In some examples, the predefined criterion may be a predetermined threshold. In some examples, the predefined criterion may be the selection of a recipe that has a temporal odour profile closest to the target temporal odour profile.

Finally, the at least one selected recipe is provided, which is usable for production of a fragrance composition with a matrix (e.g. solvents, substrates, or mixtures of solvents and substrates) or without any matrix (e.g. a perfume oil without matrix such as ethanol/h2o).

In some examples, the at least one selected recipe may be provided e.g. to a graphical user interface, to a printing unit for printing the at least one selected recipe, and/or to a storing unit for storing the at least one selected recipe.

In some examples, a control file may be generated based on the at least one selected recipe. The control file may be used for controlling the production of the fragrance composition.

The selected recipe(s) may represent a fragrance composition with the desired temporal odour profile. In this way, various aroma ingredients may be checked objectively to validate customer requirements of olfactory characteristics, to validate recipes before production/delivery and to tailor chemical products to the needs of customers. Thus, the evaluation does not rely on the subjective impact for test persons or other experimental data.

Further benefits of the proposed method may include one or more of the following:
The selected recipe(s) may be used in scent design for consumer products, fine fragrances, aroma chemicals and life style products.
The selected recipe(s) may be used for percepting, monitoring, and eliminating malodours and masking bad smell.
The proposed computer-implemented method may be suitable for tailoring odour profiles of a fragrance product based on customer's needs.
The proposed computer-implemented method may be used for exchanging ingredients in a fragrance product, which are blocked due to competitive intellectual property rights, regulatory issues in different countries or lack of resources.
The proposed computer-implemented method may reduce development costs by providing a fragrance composition with the desired temporal odour profile so that time and cost consuming reassessments of fragrance compositions can be significantly reduced.

According to an embodiment of the present invention, the fragrance product further comprises a matrix. Each recipe further comprises matrix data associated with the matrix.

In some examples, the matrix may comprise solvents, which may comprise water, alcohol such as ethanol, oil, detergent or mixtures thereof.

In some examples, the matrix may comprise substrates, which may be selected from skin, textiles, paper, wood, plastics (such as polymers, plastic composites etc.), metals or composite materials.

In some examples, the matrix may comprise a mixture of solvents and substrates.

Although not directly contributing to the olfactive impression of the fragrance composition the matrix can interact with the fragrance composition via intermolecular interactions of the components of the matrix with the ingredients of the fragrance composition, thus accelerating or slowing down the evaporation of different ingredients of the fragrance composition (boosting and retarding behaviour), and thereby can alter the olfactive impression of the fragrance composition. If an ingredient, originally attributed to the matrix, turns out to contribute to the olfactive impression, it is classified as an olfactive fragrance ingredient and attributed to the fragrance composition.

According to an embodiment of the present invention, the method further comprises the steps of:
receiving a measured performance characteristic of the fragrance product produced according to the provided recipe profile, wherein the measured performance characteristic is indicative of a temporal odour profile of the produced fragrance product;
determining a distance between the temporal odour profile of the produced fragrance product and the target temporal odour profile; and
providing, based on the distance, an updated recipe profile of the fragrance product.

In other words, the proposed recipe can be checked objectively to determine whether there is any discrepancy between the calculated temporal odour profile of the proposed recipe and the actual temporal odour profile of the produced fragrance product. If there is a discrepancy therebetween, the recipe profile can be updated accordingly.

According to an embodiment of the present invention, steps c)-f) are performed in an iterative process to determine the at least one recipe.

This will be explained hereinbelow and in particular with respect to the embodiments shown in FIGS. 3 and 4.

According to an embodiment of the present invention, the target temporal odour profile comprises a plurality of evaporation regimes over the predetermined period of time, wherein in each of the plurality of evaporation regimes a fractural amount of odour families are defined.

An example of the target temporal odour profile is shown in FIG. 5 and will be explained hereinbelow.

According to an embodiment of the present invention, in step b) the plurality of fragrance ingredients is refined by selecting fragrance ingredients with a particular performance characteristic including a particular physical characteristic, a particular chemical characteristic, or a combination thereof.

The performance characteristic may relate to a physical, chemical or physio-chemical characteristic. The performance characteristic may relate to an olfactory characteristic. The performance characteristic may comprise one or more physical, chemical or physio-chemical characteristic(s) directly or indirectly related to olfactory characteristic(s) of the fragrance product.

For example, the plurality fragrance ingredients may be refined by specifying one or more of the following characteristics: particular vapour pressure, particular odour intensity, particular clogP, and particular dipole.

According to an embodiment of the present invention, in step b) the plurality of fragrance ingredients is provided by including mandatory ingredients and/or excluding undesired ingredients.

For example, it is possible for exchanging ingredients in a fragrance product, which are blocked e.g. due to competitive intellectual property rights, regulatory issues in different countries, or lack of resources.

According to an embodiment of the present invention, step d) further comprises:
d1) receiving (140a) a recipe that comprises fragrance composition data associated with one or more fragrance ingredients of the fragrance composition;
d2) providing (140b) a vapour pressure of each fragrance ingredient based on the fragrance ingredients data;
d3) determining (140c), based on the fragrance ingredients data, a time-dependent interaction coefficient of each fragrance ingredient in a condensed phase of the fragrance composition over a predetermined period of time;
d4) generating (140d), based on the provided vapour pressure and the determined time-dependent interaction coefficient, the temporal evaporation profile of the fragrance product, wherein the temporal evaporation profile is related to a time-dependent quantity associated with the evaporation behaviour of each fragrance ingredient of the fragrance product over the predetermined period of time; and d5) providing (140e) the generated temporal evaporation profile for the recipe.

According to an embodiment of the present invention, the received receipt further comprises matrix data associated with the matrix. In step d3), the time-dependent interaction coefficient of each fragrance ingredient in a condensed phase of the fragrance composition in the matrix over a predetermined period of time is determined based on the fragrance ingredients data and the matrix data.

By determining the time dependent interaction coefficients of each fragrance ingredient, the dynamic behaviour of the fragrance product including the interactions between different fragrance ingredients and/or between fragrance ingredient(s) and the optional matrix are considered in generating the temporal evaporation profile. This way the main chemical and/or physical characteristics determining the odour of the fragrance product can be determined in a dynamic way considering correlations in a time-dependent manner. Such dynamic behaviour impacts the condensed phase in such a way that the temporal evaporation profile changes.

The methods for generating or predicting temporal odour profiles or temporal evaporation profiles of a fragrance composition or product may be based on quantum chemical calculations, which include the chemical interactions of the optional matrix and allow predictions of the chemical and olfactory behaviour of new fragrance ingredients or new matrices. The method thus provides a beneficial production aid which allows adaptations of fragrance compositions to different applications or identification of possible malodours by broadening the spectrum of objectively determining olfactive characters of fragrance ingredients.

The generation of the temporal evaporation profile solves a key need in any industry concerned with odours. Odours are thus far evaluated in a subjective human based manner. Multiple humans smell the odour and give feedback. The subjective data may be to a certain extent put into objective terms by statistical methods. However, such a process can only provide a certain degree of objectivity and is limited by the data generation via human perception. The method disclosed herein overcomes this deficiency by using physical and/or chemical properties of the fragrance composition that are key factors influencing the odour of the fragrance product. This way the chemical and/or physical properties of the fragrance composition and their dynamic behaviour can be quantified in an objective manner, solving an important need in the industry to make fragrance products objectively comparable and with that to monitor product quality in the production process or to control the production process to deliver the expected results with respect to the odour perception of the fragrance product.

In particular, the monitoring and/or validation of the fragrance composition is a key factor to ensure consistent quality of a fragrance product. The temporal evaporation profile of a fragrance product or composition as disclosed herein allows for monitoring and/or controlling production processes. Fragrance product production is highly sensitive to impurities that negatively impact the odor of the produced fragrance product. The generated temporal evaporation profile allows for the first time to provide objective quality or performance parameters that are based on chemical and/or physical properties of the fragrance product and can be measured in the production processes. A comparison of the measured and the generated temporal evaporation profile or quantities derived therefrom allows not only for quality control or more reliable production but may be extended via a feedback loop which adjusts the production process, where needed.

According to an embodiment of the present invention, a composition of the gas phase and/or the condensed phase, the interaction coefficient for each fragrance ingredient, the vapour pressure for each fragrance ingredient, or the time-dependent quantity associated with the evaporation behaviour for each fragrance ingredient is determined in a time dependent manner.

For instance for a first point in time, the composition or amount of each fragrance ingredient in the gas phase and in the condensed phase is determined. Based on the determined composition or amount of each fragrance ingredient in the gas and condensed phase, the interaction coefficient, the time-dependent quantity or the relative amount of each fragrance ingredient is determined.

Hence after an initial determination of the composition of the gas phase and the condensed phase, the interaction coefficients for each fragrance ingredient, the partial vapour pressure for each fragrance ingredient, the time-dependent quantity for each fragrance ingredient the fractural amount for each fragrance ingredient, the composition of the gas phase and the condensed phase, the interaction coefficients for each fragrance ingredient, the partial vapour pressure for each fragrance ingredient, the time-dependent quantity for each fragrance ingredient the fractural amount for each fragrance ingredient may be updated by evolving the composition of the gas phase and the condensed phase in time. This way the changes in activity for each fragrance ingredient may be accounted for, as the mixture of the composition changes over time because of the distinct evaporation of the ingredients into the gas phase. Such a temporal altering condensed phase composition significantly influences intermolecular interactions. This important fact is accounted for by updating the interaction coefficients of every ingredient along the time axis.

In step d2) the vapour pressure of each fragrant ingredient may be determined at a certain temperature, pressure and relative humidity using a chemical potential of each fragrant ingredient in the condensed phase and the gas phase. In other words, the vapour pressures of each neat fragrance ingredient may be calculated under certain conditions of temperature, pressure and/or relative humidity, usually applying ambient conditions of 15-25° C. temperature, atmospheric pressure and 40-60% relative humidity. However, also different conditions at higher or lower temperature, pressure or relative humidity may be applied. For example, the vapour pressure of each fragrant ingredient may be determined at a certain temperature, pressure and relative humidity using the chemical potentials of each fragrant ingredient in the condensed phase and the gas phase obtained by COSMO-RS or COSMO-SAC.

In some examples, in step d5) the temporal evaporation profile is related to a time-dependent fractural amount of each fragrance ingredient in the gas phase and/or the condensed phase of the fragrance composition in the matrix over the predetermined period of time.

The generated temporal evaporation profile may be usable for monitoring quality of the fragrance product in a production process and/or for validating the production of the fragrance product based on at least one precursor, such a new matrix component or fragrance ingredient, substrate. The temporal evaporation profile may be used for monitoring quality of the fragrance product in a production process and/or for validating the production quality of the fragrance product based on at least one precursor and/or substrate. Validating the fragrance composition or product may be based on the temporal evaporation profile associated with at least one new precursor and/or at least one substrate. The generated temporal evaporation profile may be provided for validating and/or monitoring the fragrance product or composition with respect to the performance characteristic. The generated temporal evaporation profile may be provided to validate and/or monitor the fragrance product or composition with respect to the performance characteristic. The generated temporal evaporation profile, which when provided to a validation apparatus, may validate and/or monitor the fragrance product or composition with respect to the performance characteristic.

According to an embodiment of the present invention, the computer-implemented method further comprises generating a control file based on the recipe profile of the at least one selected recipe, which is usable for controlling production of the fragrance product.

The term “control file” is understood to be any binary file, data, signal, identifier, code, image, or any other machine-readable or machine-detectable element useful for controlling a machine or device, for example an apparatus for monitoring production of a fragrance product, and an apparatus for validating production of a fragrance product as described herein.

For example, the control file may be used to control the dosing equipment shown in FIGS. 12 and 13 for dosing of different components of the fragrance product in the production process.

According to a second aspect of the present invention, there is provided a method for monitoring production of a fragrance product, the method comprising the steps of:
providing a target temporal evaporation profile;
providing a performance characteristic of a produced fragrance product that has a recipe profile generated according to the method of the first aspect and any associated example; and
comparing the performance characteristic with the target temporal evaporation profile to determine if the produced fragrance product fulfils predetermined quality criteria.

A comparison of the measured and the generated temporal evaporation profile or quantities derived therefrom allows not only for quality control or more reliable production but may be extended via a feedback loop which adjusts the production process, where needed.

This will be explained in detail hereinbelow and in particular with respect to the embodiments shown in FIGS. 10 and 12.

According to a third aspect of the present invention, there is provided a method for validating production of a fragrance product, the method comprising the steps of:
providing (234) an existing temporal evaporation profile for a fragrance composition that has been produced from validated precursors;
generating (236) a recipe profile based on the existing temporal evaporation profile according to the method of the first aspect and any associated example, wherein the recipe profile comprises an ingredient identifier and related property data, which are associated with at least one new precursor; and
comparing a performance characteristic of a fragrance product produced using the recipe profile and the existing temporal evaporation profile to validate the at least one new precursor.

This will be explained in detail hereinbelow and in particular with respect to the embodiments shown in FIGS. 11 and 13.

According to another aspect of the present invention, there is provided an apparatus for generating a recipe profile of a fragrance product having a target temporal odour profile, wherein the fragrance product comprises a fragrance composition with one or a plurality of fragrance ingredient(s) and a matrix, the apparatus comprising one or more processing unit(s) configured to generate the recipe profile of the fragrance product, wherein the processing unit(s) include instructions, which when executed on the one or more processing unit(s) execute the method steps according to the first aspect and any associated example.

According to another aspect of the present invention, there is provided an apparatus for monitoring production of a fragrance product, the apparatus comprising one or more processing unit(s) configured to monitor production, wherein the processing unit(s) include instructions, which when executed on the one or more processing unit(s) execute the method steps according to the second aspect and any associated example.

According to another aspect of the present invention, there is provided an apparatus for validating production of a fragrance product, the apparatus comprising one or more processing unit(s) configured to monitor production, wherein the processing unit(s) include instructions, which when executed on the one or more processing unit(s) execute the method steps according to the third aspect and any associated example.

According to a further aspect of the present invention, there is provided a computer program element comprising instructions, which when executed by a processing unit, cause the processing unit to carry out the steps of the method of the first or second aspect and any associated example.

According to another aspect of the present invention, there is provided use of recipe profile generated in a method according to the first aspect and any associated example for quality control and/or verification purposes.

The term “fragrance composition” as used herein may refer to any kind of composition comprising a plurality of volatile ingredients, which contribute to an olfactive impression, caused by interaction with the olfactory receptor cells in the olfactory epithelium of the nasal cavity of humans or animals. The olfactive impression can be a pleasant impression, qualifying the odour as pleasant odour, or an unpleasant odour, qualifying the odour as malodour. A misodour or off-odour is an odour, which was not intended. If a fragrant ingredient is odourless (i.e. not contributing to the olfactive impression) it is attributed to the matrix.

The term “matrix” as used herein may refer to a substance, which is in contact with the fragrance composition but itself does may not contribute or may be irrelevant for the purpose of the product to the olfactive impression of the fragrance composition. The matrix can be liquid or solid and can be selected from solvents, which comprise water, alcohol such as ethanol, oil, detergent or mixtures thereof, or substrates, which are selected from skin, textiles, paper, wood, plastics (such as polymers, plastic composites etc.), metals or composite materials, or mixtures of solvents and substrates. Although not directly contributing to the olfactive impression of the fragrance composition the matrix can interact with the fragrance composition via intermolecular interactions of the components of the matrix with the ingredients of the fragrance composition, thus accelerating or slowing down the evaporation of different ingredients of the fragrance composition (boosting and retarding behaviour), and thereby can alter the olfactive impression of the fragrance composition. If an ingredient, originally attributed to the matrix, turns out to contribute to the olfactive impression, it is classified as an olfactive fragrance ingredient and attributed to the fragrance composition.

The “molecular geometry” of a chemical molecule, especially a fragrance ingredient, as used herein may refer to the three-dimensional shape of the molecule based on its orientation due to the intramolecular interactions of the covalent bonds and the intermolecular interactions, such as hydrogen bonding, ionic bonding, dipole forces or van der Waals forces, with other molecules, especially molecules of the matrix. The molecular geometry is constituted as a function of spatially dependent electron density and can be calculated using quantum chemical models/methods such as density functional theory (DFT), Hartree-Fock (HF), post-Hartree-Fock or semi-empirical quantum chemical models.

The term “vapour pressure”, also referred to as equilibrium vapour pressure, as used herein, may refer to the pressure exerted by a vapour in thermodynamic equilibrium with its condensed phases (solid or liquid) at a given temperature in a closed system. The equilibrium vapour pressure is an indication of a liquid's evaporation rate. It relates to the tendency of particles to escape from the liquid (or a solid).”

### BRIEF DESCRIPTION OF THE FIGURES

In the drawings, like reference characters generally refer to the same parts throughout the different views. Also, the drawings are not necessarily to scale, emphasis instead generally being placed upon illustrating the principles of the invention.

FIG. 1 illustrates a block diagram of an exemplary device for generating a recipe profile.

FIG. 2 illustrates a flow chart describing a computer-implemented method for generating a recipe profile of a fragrance product having a target temporal odour profile.

FIG. 3 illustrates another example of the computer-implemented method.

FIG. 4 illustrates a further example of the computer-implemented method.

FIG. 5 illustrates a flow chart describing a computer-implemented method for determining a temporal odour profile of a proposed recipe.

FIG. 6 illustrates a further example describing a computer-implemented method for determining a temporal odour profile of a proposed recipe.

FIG. 7 illustrates the temporal evaporation profile of a perfume composition X predicted with the method of the invention with the fragrance ingredients shown in the list on the left side together with their main odour descriptions. The temporal olfactive profile is summarized into an odour description of the top note, core and bottom note.

FIG. 8 illustrates the effect of exchanging one ingredient in a recipe of a perfume composition on the temporal evaporation profile predicted by the method of the disclosure.

FIG. 9 illustrates the Odour Description Wheel, proposed by McGinley & McGinley (2002), Odour testing biosolids for decision-making.

FIG. 10 shows an example of a flowchart for monitoring quality of the fragrance product in a production process of the fragrance product having a target temporal evaporation profile.

FIG. 11 shows an example of a flowchart for validating the production of the fragrance product.

FIG. 12 shows an example of a production line for producing the fragrance product with a monitoring apparatus.

FIG. 13 shows another example of a production line for producing the fragrance product with a validation apparatus.

FIG. 14 shows discrete areas of odour descriptions obtained from a sketched temporal odour-profile.

FIG. 15 shows an example of a predicted recipe obtained from the odour descriptions of FIG. 14.

### DETAILED DESCRIPTION OF THE INVENTION

Although the following detailed description is described with respect to the generation of a recipe profile of a fragrance product having a matrix for the purposes of illustration, anyone of ordinary skill in the art will appreciate that the matrix is optional, and the apparatus and method described above and below can be adapted to the generation of a recipe profile of a fragrance product without any matrix, such as perfume oils without matrix such as ethanol/h2o. Accordingly, the following described examples are set forth without any loss of generality to, and without imposing limitations upon, the claimed invention.

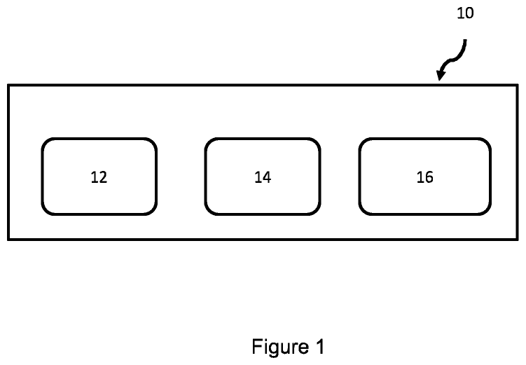

FIG. 1 illustrates a block diagram of an exemplary device 10 for generating a recipe profile of a fragrance product having a target temporal odour profile.

The device 10 may include one or more processing units 12. Optionally, as shown in FIG. 1, the device 10 may include a memory 14, and one or more communications modules 16.

In general, the device 10 may comprise various physical and/or logical components for communicating and manipulating information, which may be implemented as hardware components (e.g., computing devices, processors, logic devices), executable computer program instructions (e.g., firmware, software) to be executed by various hardware components, or any combination thereof, as desired for a given set of design parameters or performance constraints. Although FIG. 1 may show a limited number of components by way of example, it can be appreciated that a greater or a fewer number of components may be employed for a given implementation.

In some examples, the device 10 may be implemented by a computing platform such as a mobile platform, personal computer (PC) platform, and/or consumer electronics (CE) platform supporting various networking, communications, and/or multimedia capabilities. Such capabilities may be supported by various networks, such as a Wide Area Network (WAN), Local Area Network (LAN), Metropolitan Area Network (MAN), wireless WAN (WWAN), wireless LAN (WLAN), wireless MAN (WMAN), wireless personal area network (WPAN), Worldwide Interoperability for Microwave Access (WiMAX) network, broadband wireless access (BWA) network, the Internet, and/or any other wired or wireless network in accordance with the described embodiments.

In some implementations, the device 10 may comprise a system within and/or coupled to a computing device such as PC, desktop PC, notebook PC, laptop computer, mobile internet device (MID), mobile computing device, smart phone, personal digital assistant (PDA), mobile telephone, or other type of computing device in accordance with the described embodiments. The computing device may include, for example, an electronic display.

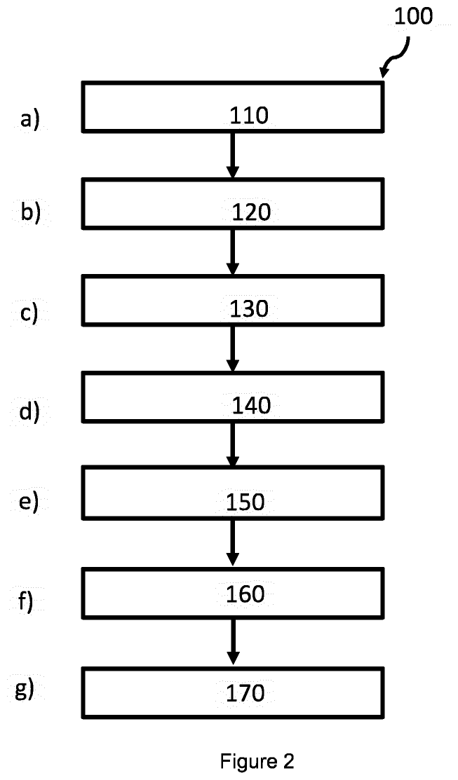

The processing unit(s) 12 may execute instructions to perform the method described herein, which will be explained in detail with respect to the embodiments shown in FIG. 2.

The memory 14 may include, but is not limited to, volatile memory and/or non-volatile memory. The memory 14 may be used to store processor instructions, and other data and instructions to enable the processor to perform the techniques described herein.

The one or more communications modules 16 may include hardware and/or software to enable the device 10 to receive a user input that defines the target temporal odour profile, and to communicate with other devices and/or a network. For example, the one or more communications modules 16 may receive the user input via a wired connection or via a wireless connection. The one or more communications modules 16 may also provide cellular telephone communications, and/or other data communications for the device 10.

FIG. 2 illustrates a flow chart describing a computer-implemented method 100 for generating a recipe profile of a fragrance product having a target temporal odour profile, in accordance with an embodiment.

Beginning at block 110 i.e. step a), the target temporal odour profile is provided e.g. to the device 10 shown in FIG. 1. The target temporal odour profile comprises a time-dependent fractural amount of a plurality of odour families over a predetermined period of time, which is indicative of a desired evaporation behaviour of each odour family in the fragrance composition.

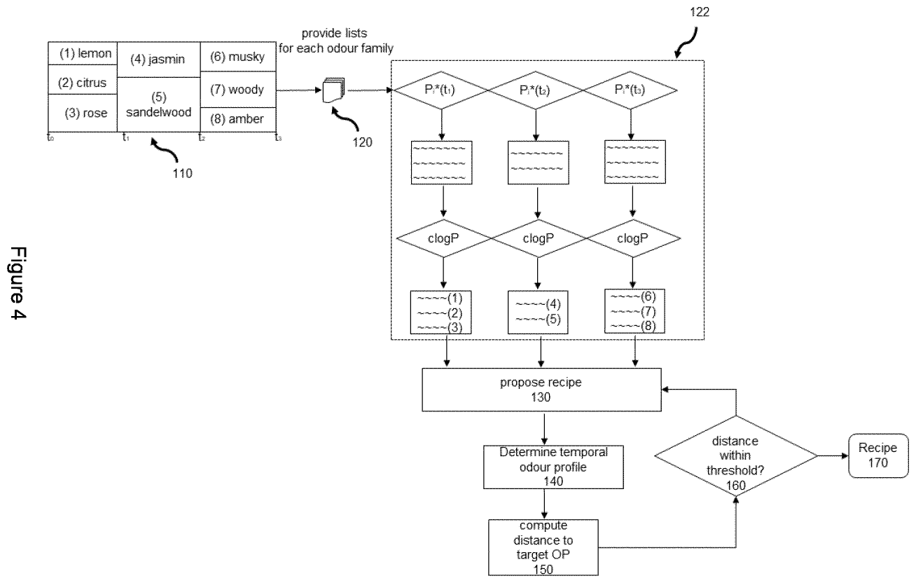

For example, the target temporal odour profile comprises a time-dependent fractural amount of a plurality of odour families in a gas phase over a predetermined period of time. In an example, the target temporal odour profile may be defined as a plurality of evaporation regimes over the predetermined period of time. Each evaporation regime corresponds to a time segment in the predetermined period of time. Different time segments have different evaporation profile and are therefore referred to as evaporation regimes. The fractural amount of each odour family may be represented by an area in the evaporation regimes, which may also be referred to as olfactory area. An example of the target temporal odour profile is shown in FIG. 4 and will be discussed hereinbelow.

At block 120, i.e. step b), for each odour family, one or more fragrance ingredients are provided that have olfactive contributions matching the respective odour family.

The plurality of fragrance ingredients may be retrieved from a database (DB). From many fragrance ingredients, descriptions of the odour families and their contributions to the total olfactive perceptions are already known and can be retrieved from databases. Such databases can be commercially available databases such as e.g. ScenTree, Flavornet, GoodScents, SuperScent or Sigma-Aldrich, or an internal database. Such a database usually lists the name and the structure of the fragrance ingredient together with several descriptions of the main and sub-odour families and optional additional attributes of the fragrance ingredient, such as e.g. molecular weight or boiling points, if available. Usually the number of descriptions of the fragrance ingredient varies from about 4 to 10.

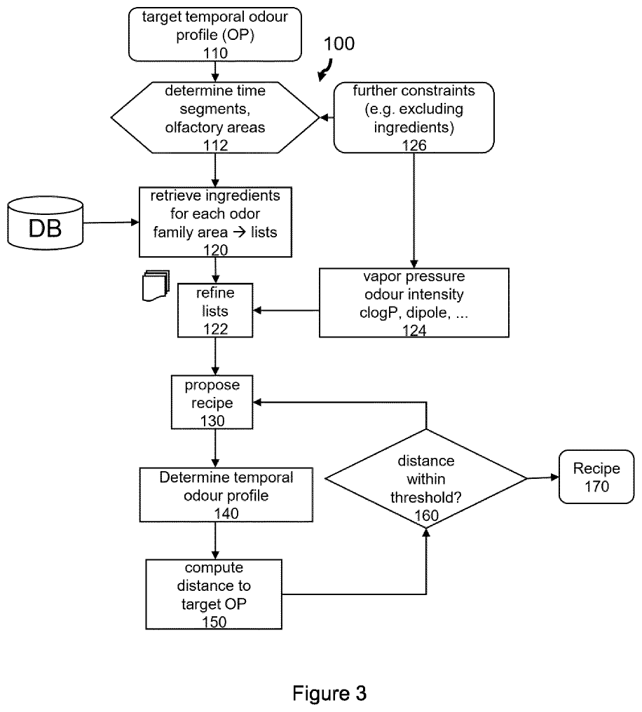

Optionally, the list of fragrance ingredients may be further refined. This will be explained hereinbelow and particularly with respect to the examples shown in FIGS. 3 and 4.

At block 130, i.e. step c), at least one ingredient is selected from each odour family to form one or more recipes of the fragrance product. Each recipe comprises fragrance composition data associated with the fragrance ingredients of the recipe and matrix data associated with the matrix. The fragrance composition data may be associated with one or more fragrance ingredients of the fragrance composition. The fragrance composition data may include a fragrance ingredient identifier for each fragrance ingredient and an absolute or relative amount for each fragrance ingredient initially present in the composition. The fragrance ingredient identifier may include a representation of the molecule. The fragrance ingredient identifier may be associated with a representation of the molecular structure and/or the molecular geometry. This way the fragrance ingredient data may specify the composition of the fragrance composition.

All fragrance ingredients in a recipe may be defined by listing them and defining their amounts in the recipe. A fragrance ingredient thereby can be a single chemical substance or a combination of chemical substances. The fragrance ingredients can have natural, semi-synthetic or synthetic origin. It is also possible to use a sub-formula, i.e. a combination of fragrance ingredients that are used in a fix ratio, and to add such a sub-formula as one ingredient. The recipe is thereby defined as the sum of all fragrance ingredients without solvent, thus the sum of all fragrance ingredients making up 100 wt % of the recipe. For a perfume composition for example, the recipe is reflected by the pure perfume oil.

Components of the matrix adding to the olfactive impression are attributed to the recipe. Fragrance ingredients not adding to the olfactive impression can either be attributed to the matrix or to the recipe and thereby subjected to the method of the present disclosure for identifying their good or bad non-odour effects in the temporal evaporation profile, such as e.g. balming effects or caustic or toxic effects.

Further, the matrix data may be associated with the matrix or the matrix material. The matrix data may include components identifiers associated with one or more matrix components. The matrix may be liquid or solid. In some examples, it is selected from solvents, which comprise water, alcohol, oil, detergent or mixtures thereof, or substrates. In some examples, it is selected from substrates, such as skin, textiles, paper, wood, plastics, such as polymers or plastic composite materials, metals or composite materials. In some examples, it is selected from mixtures of solvents and substrates.

Depending on the application of the recipe, such as e.g. a perfume oil, the matrix may be a solvent, such as ethanol, water, oil or detergent or mixtures thereof used for the preparation of e.g. an eau de parfum, eau de toilette, a hair shampoo, a shower or washing lotion, a soap or a body cream. When defining a solvent or mixture of solvents as matrix the amount of the solvent(s) usually are also defined.

The fragrance ingredients, their amounts and the matrix may be defined by preparing a list of the names of the selected ingredients and matrix components usually together with their relative proportions such as e.g. in a spread sheet.

Each recipe may differ from one another in the composition of fragrance ingredients and/or the corresponding fractural amounts. In an example, two recipes may have the same fragrance ingredients but with different fractural amounts. In another example, two recipes may have one or more different fragrance ingredients. These fragrance ingredients are selected from the list of fragrance ingredients provided at block 120.

In one option, the plurality of recipes may be provided in an iterative process. For example, an initial recipe is provided. If the temporal odour profile for the initial recipe does not satisfy a redefined criterion. The initial recipe may be adjusted by changing one or more fragrance ingredients in the initial recipe and/or the fractural amount of one or more of the fragrance ingredients in the initial recipe. This process may be repeated until an updated recipe has a temporal odour profile that satisfies the predefined criterion. This will be explained hereinbelow and particularly with respect to the example shown in FIGS. 3 and 4.

In another option, the plurality of recipes may be provided in a non-iterative process. For example, multiple recipes may be proposed with each recipe differing from one another in the composition of fragrance ingredients and/or the corresponding fractural amounts. The temporal odour profiles of all recipes are then determined. At least one recipe that has a temporal odour profile satisfying the predefined criterion may be selected from the multiple proposed recipes.

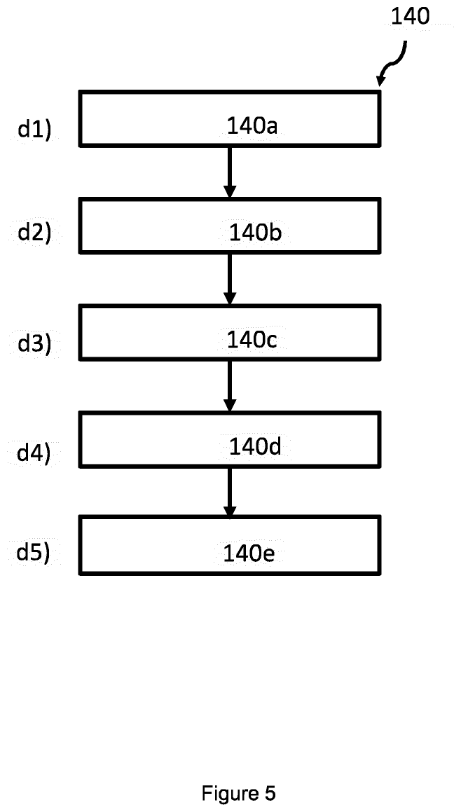

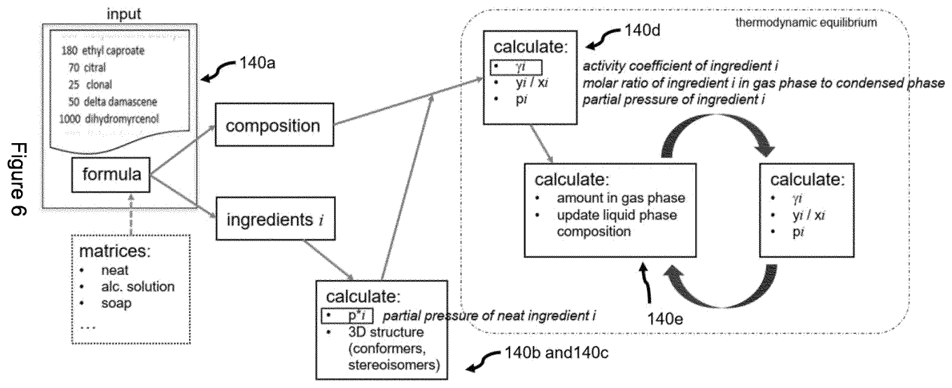

At block 140, i.e. step d), a temporal odour profile for each of the one or more recipes is determined. This will be explained hereinbelow and in particular with respect to the examples shown in FIGS. 5 and 6.

At block 150, i.e. step e), a distance of each determined temporal odour profile of the one or more recipes to the target temporal odour profile of the fragrance product is determined.

For example, the determined temporal odour profile may also be divided into a plurality of evaporation regimes similar to that of the target temporal odour profile. The fractural amounts of the odour families in each evaporation regime of the determined temporal odour profile may be compared with that of the target temporal odour profile to determine the distance.

Optionally, as shown in FIG. 3, steps c) to e) may be performed in an iterative process.

At block 160, i.e. step f), at least one recipe is selected from the one or more recipes that has a distance satisfying a predefined criterion. In some examples, the predefined criterion may be a predetermined threshold. In some examples, the predefined criterion may be the selection of a recipe that has a temporal odour profile closest to the target temporal odour profile.

At block 170, i.e. step g), a recipe profile of the at least one selected recipe preferably usable for production of the fragrance product.

In some examples, the recipe profile may be provided e.g. to a graphical user interface, to a printing unit for printing the at least one selected recipe, and/or to a storing unit for storing the recipe profile.

In some examples, a control file may be generated based on the recipe profile. The control file may be used for controlling the production of the fragrance product.

FIG. 3 illustrates another example of the computer-implemented method 100. At block 110, a user input is received that defines a target temporal odour profile. The target temporal odour profile comprises a time-dependent fractural amount associated with an evaporation behaviour of a plurality of odour families over a predetermined period of time.

At block 112, the time segments and olfactory areas are determined. As discussed hereinbefore, the time segments may also be referred to as evaporation regimes. Each olfactory area represents a fractural amount of a particular odour family in an evaporation regime. An example is shown in FIG. 4 and will be discussed hereinafter.

At block 120, fragrance ingredients are retrieved from a database, such as ScenTree, Flavornet, GoodScents, SuperScent or Sigma-Aldrich, or an internal database, based on the descriptions of the main and sub-odour families of fragrance ingredients in the database.

At block 122, the list of fragrance ingredients may be refined by selecting fragrance ingredients according to one or more attributes of the fragrance ingredients. For example, the list of fragrance ingredients may be selected according to one or more of the following attributes including, but are not limited to, the vapour pressure, odour intensity, clogP, and dipole as shown in block 124 of FIG. 3. Additionally or alternatively, the list of fragrance ingredients may be refined by including mandatory ingredients and/or excluding undesired fragrance ingredients as shown in block 126 of FIG. 3.

At block 130, an initial recipe is proposed with fragrance ingredients selected from the refined list of fragrance ingredients and an initial fractural amount of each fragrance ingredient.

At block 140, a temporal odour profile is determined for the initial recipe, for example, according to the example shown in FIG. 5.

At block 160, a distance of the temporal odour profile for the initial recipe to the target odour profile is determined.

At block 162, the determined distance is compared with a predetermined threshold. If the determined distance is within the predetermined threshold, the initial recipe is selected (block 170). Otherwise, the proposed initial recipe is adjusted by changing one or more fragrance ingredients in the initial recipe and/or a fractural amount of one or more fragrance ingredients in the initial recipe (block 130). This process may be repeated until an updated recipe has a temporal odour profile with a distance to the target temporal odour profile within the predetermined threshold. Then, the updated recipe is selected at block 170 for the production of the fragrance composition.

FIG. 4 illustrates a further example of the computer-implemented method 100. At block 110, a user input that defines a target temporal odour profile is received. In this example, the predetermined period of time are divided into three time segments including time segments t₀-t₁, t₁-t₂, and t₂-t₃. These time segments have different evaporation profiles, and are also referred to as evaporation regimes. In each evaporation regimes, a fractural amount of odour families are defined. In the evaporation regime t₀-t₁ of FIG. 4, three odour families are defined, namely lemon, citrus, and rose. The area occupied by each odour family represents its fractural amount in this evaporation regime. In the evaporation regime t₁-t₂ of FIG. 4, two odour families are defined, namely jasmin and sandelwood. Similarly, the area occupied by each odour family represents its fractural amount in this evaporation regime. In the evaporation regime t₂-t₃ of FIG. 1, three odour families are defined, namely musky, woody, and amber. Likewise, the area occupied by each odour family represents its fractural amount in this evaporation regime.

At block 120, fragrance ingredients, such as musky and sandelwood, are retrieved from a database based on the descriptions of the main and sub-odour families of fragrance ingredients in the database. For each odour family, a list of fragrance ingredients may be compiled.

At block 122, the lists of fragrance ingredients are refined by selecting fragrance ingredients based on one or more desired attributes. In the example shown in FIG. 4, the lists of fragrance ingredients are refined by selecting fragrance ingredients based on vapour pressure and clogP.

At block 130, an initial recipe is proposed with fragrance ingredients selected from the refined list of fragrance ingredients and an initial fractural amount of each fragrance ingredient.

At block 140, a temporal odour profile is determined for the initial recipe, for example, according to the example shown in FIG. 5.

At block 150, a distance of the temporal odour profile for the initial recipe to the target odour profile is determined.

At block 160, the determined distance is compared with a predetermined threshold. If the determined distance is within the predetermined threshold, the initial recipe is selected (block 170). Otherwise, the proposed initial recipe is adjusted by changing one or more fragrance ingredients in the initial recipe and/or a fractural amount of one or more fragrance ingredients in the initial recipe (block 130). This process may be repeated until an updated recipe has a temporal odour profile with a distance to the target temporal odour profile within the predetermined threshold. Then, the updated recipe is selected for the production of the fragrance composition.

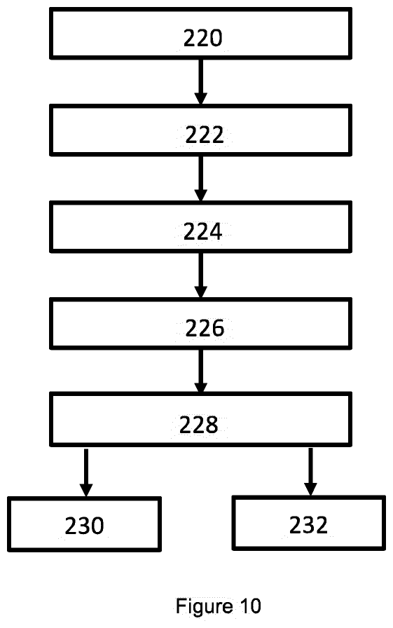

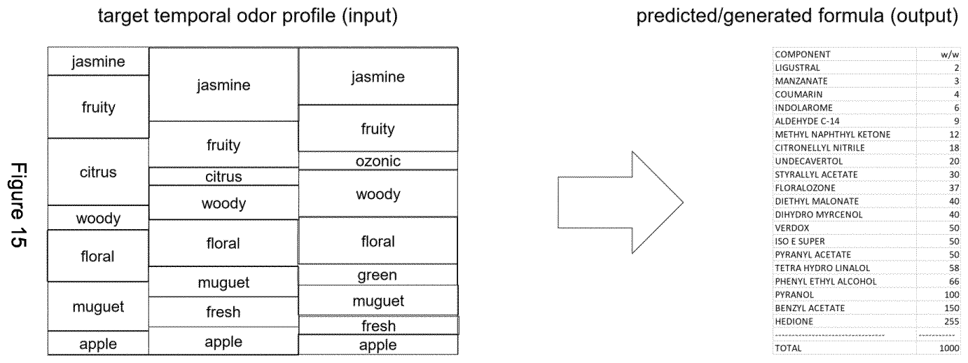

In FIG. 4, block 110 starts from receiving a user input that defines a target temporal odour profile in form of a table. However, it will be appreciated that the user input may define the target temporal odour profile in a different form. For example, as shown in FIG. 14, block 110 may start from receiving a human-sketched temporal odour-profile which shows a desired odour profile. An example of the human-sketched temporal odour-profile is shown in the left figure of FIG. 14. The sketched temporal odour-profile may be simplified and comprise now discrete areas of odour descriptions (e.g., summing up to 1 or 100% on y-axis) and representing top notes, middle notes, and dry down on the time or x-axis. The obtained simplified and discretized odour profile shown in the right figure of FIG. 14 marks the input of the program that predicts suited recipes. As shown in FIG. 15, the input is then fed to a program and is processed according to the method disclosed herein, e.g., the method 100 shown in FIG. 4, to obtain predicted recipe or formula, e.g., the formula shown in the right figure of FIG. 15. The predicted recipe shown in FIG. 15 comprises a list of the names of the determined ingredients and matrix components together with their relative proportions as measured by weight or mass, such as weight by weight (w/w) shown in FIG. 15. A control file may be generated based on the predicted recipe or formula for controlling production of the fragrance product, e.g., for monitoring production of a fragrance product and validating production of a fragrance product as described hereinafter and in particular with respect to the examples shown in FIGS. 10 to 13.

FIG. 5 shows an example of a flowchart a flow chart describing the determination of a temporal odour profile for a proposed recipe, i.e. block 140 of the computer-implemented method 100 shown in FIG. 2.

In step 140a, i.e. step d1), a proposed recipe is provided. The proposed recipe may be an initial recipe or an updated recipe in an iterative process. The proposed recipe may also be referred to as fragrance composition. As described above, the recipe comprises fragrance composition data and matrix data. The fragrance composition data may be associated with one or more fragrance ingredients of the fragrance composition. The fragrance composition data may include a fragrance ingredient identifier for each fragrance ingredient and an absolute or relative amount for each fragrance ingredient initially present in the composition. The fragrance ingredient identifier may include a representation of the molecule. The fragrance ingredient identifier may be associated with a representation of the molecular structure and/or the molecular geometry. This way the fragrance ingredient data may specify the composition of the fragrance composition. The matrix data may be associated with the matrix or the matrix material. The matrix data may include components identifiers associated with one or more matrix components.

Molecular geometry data related to one or more fragrance ingredient(s) may be provided. Such molecular geometry data may include a representation of the molecular geometry of the respective fragrance ingredient. The molecular geometry data may include a representation of the molecular geometry of the respective fragrance ingredient independent or dependent on the matrix data.

The molecular structure and/or molecular geometry data of the respective fragrance ingredient may be provided in form of a chemical file format specifying molecules. In particular the molecular structure and/or molecular geometry representation may specify atoms, bonds, coordinates and/or further property data associated with the molecule. Chemical file formats may comprise computational data formats for coding chemical information. Examples are SMILES, XYZ, MDL, SDF or MOL as described below.

The Simplified Molecular Input Line Entry Specification (SMILES) may be a line notation for molecules. SMILES strings include connectivity but do not include 2D or 3D coordinates.

The XYZ format is a simple format that usually gives the number of atoms in the first line, a comment on the second, followed by a number of lines with atomic symbols (or atomic numbers) and cartesian coordinates. The MDL number contains a unique identification number for each reaction and variation. The format is RXXXnnnnnnnn. R indicates a reaction, XXX indicates which database contains the reaction record. The numeric portion, nnnnnnnn, is an 8-digit number. SDF and MOL are other file formats from MDL Information Systems. The molfile consists of some header information, the Connection Table (CT) containing atom info, then bond connections and types, followed by sections for more complex information. “SDF” stands for structure-data file, and SDF files actually wrap the molfile format. Multiple compounds are delimited by lines consisting of four dollar signs ($$$$). A feature of the SDF format is its ability to include associated data such as e.g. molecular weight.

In step 140b, i.e. step d2), vapour pressure data related to one or more fragrance ingredient(s) may be provided. Such vapour pressure data may include the vapour pressure associated with the respective fragrance ingredient. The vapour pressure may relate to the vapour pressure based on the molecular structure and/or geometry of the respective fragrance ingredient. The vapour pressure may relate to the vapour pressure based on the molecular structure and/or geometry of the respective fragrance ingredient independent of the matrix, dependent on the matrix or both.

In step 140c, i.e. step d3), a time dependent interaction coefficient may be determined for each fragrance ingredient in the condensed phase of the fragrance composition in the matrix over a predetermined period of time. Such time dependent interaction coefficient may be determined based on the provided fragrance ingredients data and the matrix data.

The interaction coefficient may link ideal behaviour of the fragrance ingredient with real behaviour the fragrance ingredient in condensed phase. The interaction coefficient may thus include interactions between fragrance ingredient-fragrant ingredient and fragrant ingredient-matrix.

In addition to the interactions, changes in the fragrance composition (e.g. odorous oils) in the matrix over time e.g. due to different evaporation behaviour of components, can be included by updating the composition in the gas and/or condensed phase and determining the interaction coefficient for such updated composition. The activity coefficients of each fragrance ingredient in a given mixture at t0 and t1-tx may be calculated utilizing a quantum-chemical model, such as the conductor-like screening model for real solvents (COSMO-RS) that is based on chemical potentials in condensed phase of the fragrance composition in a matrix. Alternatively, these activity coefficients may be calculated based on a thermodynamic model, such as UNIQUAC, thereby utilizing group-contribution methods, such as UNIFAQ.

The activity coefficients of every ingredient in the mixture may be calculated in the initially (t0). From the calculated vapour pressure and interaction coefficient of each fragrant ingredient, the composition of the gas phase and the condensed phase of the fragrance composition is determined at the initial time t0. The activity coefficients may be updated in certain intervals over time (t1-tx) as the composition of the fragrance ingredients in the matrix of the condensed phase alters due to distinct evaporation of fragrant ingredients from the condensed phase into the gas phase. The activity coefficients hence allow to consider changes, as the mixture of the composition changes over time because of the distinct evaporation of the fragrance ingredients into the gas phase. Such a temporal altering of the condensed phase composition influences intermolecular interactions. This important fact is accounted for by updating the activity coefficients of every ingredient along the time axis. This is preferably done on-the-fly in the backend of the program.

In step 140d, i.e. step d4), the temporal evaporation profile of the fragrance composition in the matrix is generated based on the vapour pressure data and the determined time-dependent activity coefficient. The temporal evaporation profile may relate to a time-dependent quantity associated with the evaporation behaviour of each fragrance ingredient of the fragrance product over the predetermined period of time. The temporal evaporation profile may relate to a time-dependent fractural amount of each fragrance ingredient in the gas phase and the condensed phase of the fragrance composition in the matrix over the predetermined period of time. In other words the temporal evaporation profile may indicate for each fragrance ingredient an absolute or relative amount of such ingredient in the condensed and in the gas phase. This way the fragrance product comprising the fragrance composition and the matrix material may be characterized through its dynamic evaporation characteristics. Such evaporation characteristics of the fragrance composition may correlate directly or indirectly to a performance characteristic of the fragrance product such as a performance characteristic.

In step 140e, i.e. step d5), the generated temporal evaporation profile is provided.

The generated temporal evaporation profile may be usable for validating the fragrance composition, the fragrance composition in the matrix or the fragrance product with respect to a performance characteristic is provided. The performance characteristic may relate to physical, chemical or physio-chemical characteristic. The performance characteristic may relate to an olfactory characteristic. The performance characteristic may comprise one or more physical, chemical or physio-chemical characteristic(s) directly or indirectly related to olfactory characteristic(s) of the fragrance product.

Optionally, in the generated temporal evaporation profile, the fragrance ingredients may be grouped into odour families and their contribution to the total olfactive perception.

From several fragrance ingredients descriptions of the odour families and their contribution to the total olfactive perceptions is already known and can be retrieved from databases. Such databases can be commercially available databases such as e.g. ScenTree, Flavornet, GoodScents, SuperScent or Sigma-Aldrich or an internal database. Such a database usually lists the name and the structure of the fragrance ingredient together with several descriptions of the main and sub-odour families and optional additional attributes of the fragrance ingredient, such as e.g. molecular weight or boiling points, if available. Usually the number of descriptions of the fragrance ingredient varies from about 4 to 10.

In the case that the descriptions of the odour families of a specific fragrance ingredient is not available from a database the descriptions of the odour families of said fragrance ingredient can be predicted using machine-learning algorithms. The database may comprise publicly available and internal aroma molecules with attached odour descriptions based on literature information or descriptions derived by evaluations of e.g. perfumers. To combine all molecules from different sources to odour families and odour sub-families several mapping rules were created, and the odour classes and sub-classes were harmonized. For example, the odour family “floral” may be defined by subfamilies “rose”, “lavender”, etc. In general for modelling the amount and the quality of the data may heavily influence the prediction. For this reason, it may be beneficial to develop the machine-learning model for odour families/sub-families that contain more than 50 molecules.

These algorithms are preferably trained on a computer-readable two-dimensional (2D) description of the molecular structure to identify lead structures in the molecular structure of the fragrance ingredients, which contribute to the olfactive perception of the fragrance ingredients. The prediction of the descriptions of the odour families of a fragrance ingredient using machine-learning algorithms is preferably conducted in a computer-implemented method using a processing unit comprising one or more processors as described above for the quantum chemical calculations.

One example of the machine-learning models is the random forest in the classification mode. A random forest is an ensemble of decision trees. The idea of ensemble building is that many weak classifiers may be combined to a strong one. The final classifier is much less susceptible for overfitting to the training data. This may lead to robust prediction models for unknown molecules. The molecules are classified into two classes based on a so-called prediction score. The prediction score is a class probability estimator. In general, 0.5 is the decision boundary. In this case, the two classes are “smells like a certain class” or “smell not like the certain class”. These two classes are unequally distributed—that is why a random undersampling, oversampling, and some hybrid methods were evaluated. The final model may be based on five random forest models and averaged results. In an example, the input of the machine-learning model may be a SMILES strings, and the output of the machine-learning model may be a list of predicted odour families and their probability.

In this way, the fragrance ingredients may be grouped into odour families, whereby each fragrance ingredient is usually matched with 3 to 4 descriptions, which are either obtained from a database or predicted using machine-learning algorithms. For example the fragrance ingredient “ligustral” can be attributed with the descriptions “green”, “herbaceous” and “citrus”.

Then usually about 10 to 15 odour families are defined which in the end shall be listed in the resultant temporal odour families' profile.

All amounts of the different descriptions of said defined 10 to 15 odour families may be added together. For example from 25 fragrance ingredients of the composition 5 fragrance ingredients are attributed with the description “fruity” and 3 fragrance ingredients are attributed with the description “floral”. Thereby, in a first approach each of the 3 to 4 descriptions which are selected for each fragrance ingredient is usually counted with the same weighting of olfactive perception of about 1. For example for the fragrance ingredient “ligustral” the olfactive perception of “green” is counted to be about the same as of “herbaceous” or “citrus”. The descriptions can also be weighted with different factors of olfactive perception such as e.g for the fragrance ingredient “ligustral” the olfactive perception of “green” is counted to be 1, the olfactive perception of herbaceousis counted to be 0.4 and the olfactive perception of “citrus” is counted to be 0.2.

Then, the contribution of the odour families to the total olfactive perception may be summed up according to their fractional occurrence in the gas phase per time interval.

The sum of all odour families per time interval is listed in a table and preferably normalized to 1 or 100%. An excerpt of such a table at time intervals t₁, t₂ and t₃ for 8 descriptions before normalization can be seen below. It can be seen from the numbers that the odours described with the descriptions “lavender”, “herbal” and fresh contribute the most to the olfactive perception throughout the timeframe of t₁ to t₃. The contribution of the odour described with the description “fruity” increases from t₁ to t₃, whereas the contribution of other odours such as those described with the descriptions “floral”, “galbanum”, or “green” are small but distinct.

| description | Time t₁ | Time t₂ | Time t₃ |
|---|---|---|---|
| fruity | 55443.8093 | 65242.8238 | 97911.6422 |
| floral | 1232.1055 | 1482.57624 | 2467.70061 |
| lavender | 176436.326 | 199267.005 | 248857.837 |
| bergamote | 7299.01625 | 8775.44308 | 14407.1832 |
| herbal | 140441.953 | 155891.897 | 178136.146 |
| galbanum | 220.798389 | 266.180989 | 442.890649 |
| green | 254.140349 | 306.424072 | 509.842608 |
| fresh | 140413.171 | 155857.313 | 178078.492 |

The determined temporal odour profile is then in step e), i.e. block 150 shown in FIG. 2 to determine a distance of the determined temporal odour profile of the recipe to the target temporal odour profile.

Optionally, the determined temporal odour profile may be provided e.g. to a graphical user interface, to a printing unit for printing the temporal odour profile, and/or to a storing unit for storing the temporal odour profile. For example, the temporal fragrance ingredient profile and/or the temporal odour families profile is displayed, based on the compositions of the gas phase at the predetermined times t₀, t₁-tₓ and grouping of the fragrance ingredients of the compositions of the gas phase, in order to visualize the temporal performance and olfactive perception of the fragrance composition in the matrix.

The temporal odour profiles can be displayed according to the special requirements of the user. In some examples, the temporal odour profile over time can be displayed in a graph of the fractural amounts, such as the partial pressures, of all fragrance ingredients in the gas phase of the fragrance composition over time as calculated in process step f). Thereby, the total amount of all fragrance ingredients in the gas over time is usually normalized to either 1 or 100% and the fractural amounts are listed as fractions of 1 or 100%. These temporal fractural amounts can be displayed in a graph over time in colour codes. Said option of displaying the temporal odour profile is especially suitable for displaying the volatility of each fragrance ingredient of a fragrance composition in a specific matrix and their contribution to the temporal odour profile of said fragrance composition. It can be used e.g. when comparing a fragrance composition in different matrices and how the different matrices influence the volatility of different fragrance ingredients.

In some examples, the temporal odour profile can be displayed in a graph of the fractural amounts of all odour families in the gas phase of the fragrance composition over time. Thereby, the total amount of all odour families in the gas phase of the fragrance composition over time is usually normalized to either 1 or 100% and the fractural amounts are listed as fractions of 1 or 100% as discussed above. These temporal fractural amounts can be displayed in a graph over time in colour codes. Said option of displaying the temporal odour profile is especially suitable for displaying the development of the olfactive perception of a fragrance composition in a specific matrix and determine the odour development over time such as the top note, core note and bottom note of the fragrance composition in a specific matrix. Said odour development can then be compared to the odour development over time of the same fragrant composition in a different matrix. Additionally, any possible misodours or malodours, which may develop over time can be determined.

It will be appreciated that the above operation may be performed in any suitable order, e.g., consecutively, simultaneously, or a combination thereof, subject to, where applicable, a particular order being necessitated, e.g., by input/output relations.

FIG. 6 illustrates a further example of the determination of a temporal odour profile for a proposed recipe. A fragrance composition in a matrix comprising a plurality of fragrance ingredients

The recipe is defined by introducing the ingredients together with their amounts at a block corresponding to block 140a in FIG. 5. Thereby, the ingredients are introduced in chemical file format such as in form of chemical file formats such as SMILES code, XYZ, MDL mol or SDF.

Additionally, the matrix is defined, e.g. by defining the solvent, such as ethanol, together with its amount. The chemical file formats of the ingredients are used in the processing unit for calculating the molecular geometry of each ingredient depending on the matrix using quantum chemical calculations, preferably together with its conformers and stereoisomers, thereby selecting either the best conformer or an ensemble of conformers.

The molecular geometry, preferably the best conformer or an ensemble of conformers is then used for calculating the vapour pressures of each neat fragrance ingredient using quantum chemical calculations at a block corresponding to blocks 140b and 140c in FIG. 5.

The calculated data of each fragrance ingredient and the data of the fragrance composition such as the amounts of the different ingredients are then used for calculating the partial pressure pᵢ, the time-dependent interaction coefficient γᵢ and the molar ratios xᵢ and yᵢ of each ingredient in the condensed phase and gas phase at the thermodynamic equilibrium at the beginning t₀. This corresponds to block 140d in FIG. 5.

From this data the fractural amounts of each ingredient in the gas phase at given time intervals t₁-tₓ are calculated by recalculating the partial pressure pᵢ, the time-dependent interaction coefficient γᵢ and the molar ratios xᵢ and yᵢ of each ingredient in the condensed phase and gas phase at the thermodynamic equilibrium at said time intervals t₁-tₓ based on the different composition of the condensed phase over time. This corresponds to block 140e in FIG. 5.

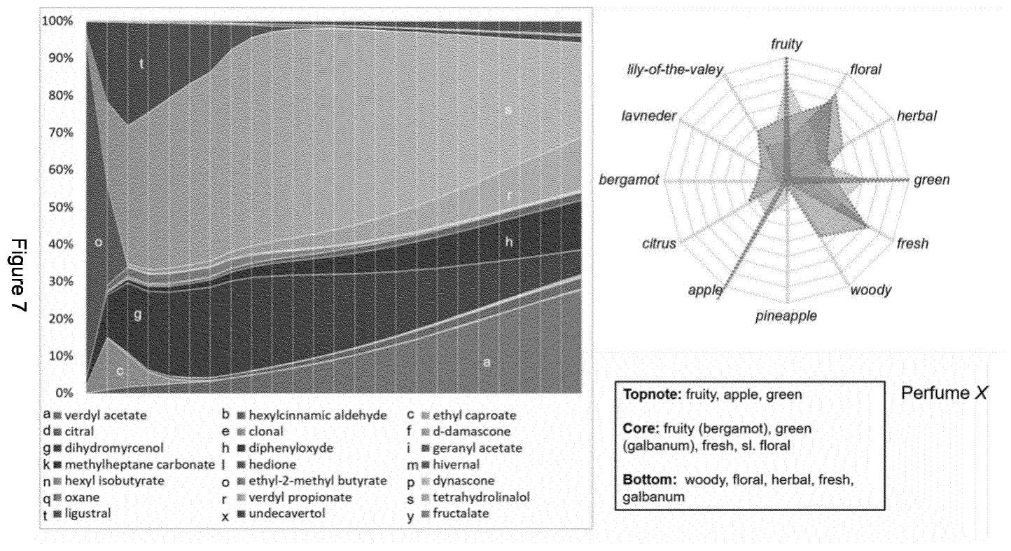

FIG. 7 shows the temporal evaporation profile of a perfume x in form of a temporal odour families' profile. The development of the fractural amounts of the different odour families in the gas phase over time are shown in colour code. The perfume includes 21 fragrance ingredients which are listed below the graph on the left side. These ingredients are grouped in 12 odour families together with their olfactive contributions shown in the odour wheel on the right side. From the graph of the temporal evaporation profile the predominant odour families over time can be determined thereby establishing a prediction of the top note, core note and bottom note of perfume x.

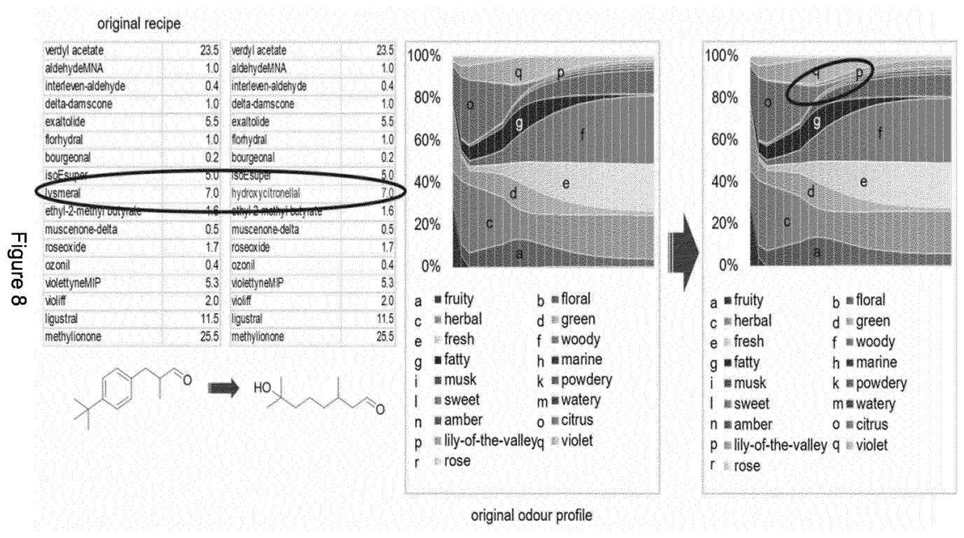

FIG. 8 shows the differences in the temporal evaporation profile of a fragrance composition when exchanging one ingredient with another. In the present case 7% lysmeral is exchanged for 7% hydroxycitronellal. No other amendment has been made to the recipe.

On the left side the temporal evaporation profile of the original recipe comprising lysmeral is shown whereas on the right side the temporal evaporation profile of the adapted recipe comprising hydroxycitronellal is shown. Both temporal evaporation profiles are shown as colour coded temporal odour families' profiles.

When comparing the two temporal evaporation profiles it can be seen that the biggest impact of the exchange to hydrocitronellal is the increase of the fractural amount of the lily-of-the-valley odour description in a middle and late time interval, which indicates a slight shift in the core note and bottom note towards a pronounced floral (lily-of-the-valley) note of the adapted recipe.

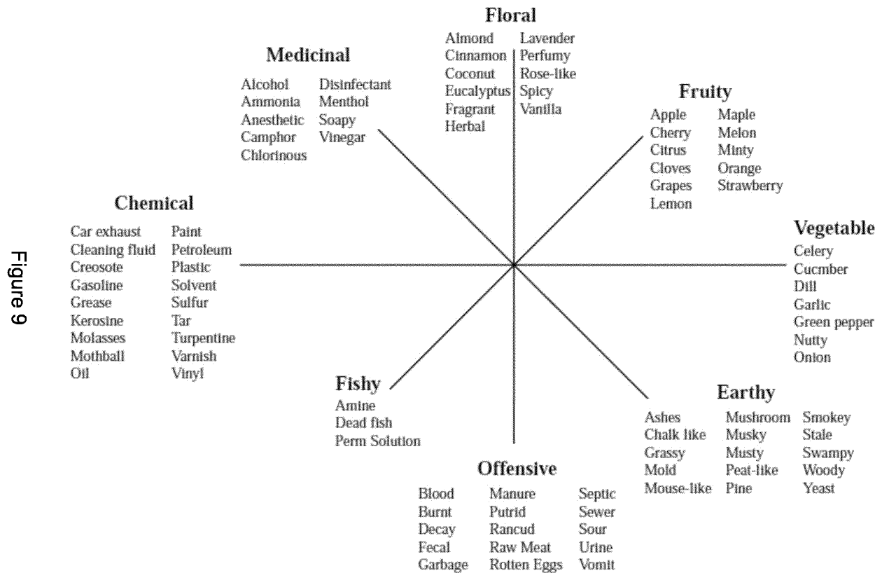

FIG. 9 shows the Odour Description Wheel, proposed by McGinley & McGinley (2002), Odor testing biosolids for decision-making. In: Water Environment Federation Specialty Conference. Residuals and Biosolids Management Conference. Austin (EUA), 3-6 as one example of an odour description wheel showing odour description main and subclasses only for illustrating purposes. Said distinct odour description wheel is shown here as illustrative example as it only includes a limited number of main and subclasses but still gives an impression on how main and subclasses are selected.

FIG. 10 shows an example of a flowchart for monitoring quality of the fragrance product in a production process of the fragrance product having a target temporal evaporation profile.

In step 220, the target temporal evaporation profile is provided e.g. from a user input.

In step 222, the performance characteristic of the produced fragrance product is provided. The produced fragrance product has a recipe profile as generated according to the method described therein.

The performance characteristic may be provided by or derived from measurement data. Such measurement data for instance includes measurement data provided by a chemical sensor. The chemical sensor may be at least partially selective with respect to different molecules. The chemical sensor may be configured to detect the presence of one or more molecule(s). The chemical sensor may be configured to detect the presence and absolute or relative amount one or more molecule(s). Chemical sensors are for instance based on electrochemical or optical sensing techniques to detect one or more molecules. Chemical sensors may detect molecules in the gas phase and/or in the condensed phase. As an example electrochemical sensors may be configured to detect a wide range of molecules. In addition their detection sensitivity may be tailored to specific molecules. As a further example infrared or photo detectors may be configured to detect a wide range of molecules. In addition their detection sensitivity may also be tailored to specific molecules. Chemical sensors may include a combination of one or more detection techniques to tailor the sensitivity of the sensor to the molecules to be detected.

In step 224, the performance characteristic as provided or measured may be compared to the target temporal evaporation profile to determine if the produced fragrance product fulfils predetermined quality criteria.

In order to enable such comparison, the evaporation profile may include one or more physical, chemical or physio-chemical characteristic(s) that relate to the performance characteristic.

In one example the evaporation profile may specify the relative amount of a fragrance ingredient in the gas phase over time. In another example the evaporation profile may specify the relative amount of a fragrance ingredient in the condensed phase over time. In yet another example the evaporation profile may specify a substantivity, a tenacity, a bloom, a boost characteristic, a retention characteristic, a note, or a burning effectiveness of a fragrance product. An exemplary target temporal evaporation profile is illustrated in FIG. 4.

The performance characteristic may include data from a measurement over time of a fragrance product's gas and/or condensed phase. It may include the relative or absolute amount of one or more molecules in the gas and/or condensed phase over time. If the evaporation profile specifies the relative or absolute amount of one or more fragrance ingredient(s) in the gas and/or condensed phase over time, the measured values may be compared to the corresponding values provided via the evaporation profile.

Optionally, in step 226, the target temporal evaporation profile may be mapped to the performance characteristic. In other word the values corresponding to the performance characteristic may be determined from target the temporal evaporation profile. In other embodiments the performance characteristic may be mapped to the target temporal evaporation profile. Both options are equally applicable.

Optionally, in step 228, the target temporal evaporation profile and the performance characteristic or any corresponding values derived therefrom are used for validation. Such validation may be performed by comparing values or value ranges.

If the values lie within an acceptable range or value, such as a 1- or 2-standard deviation(s) interval, the fragrance product as measured may be valid in the sense that it fulfils the performance criterium or criteria. If the values do not lie within an acceptable range, such as a 1- or 2-standard deviation(s) interval, the fragrance product as measured may be invalid in the sense that it does not fulfil the performance criterium or criteria.

Optionally, if the fragrance product is valid, e.g. a control signal for a production process may be triggered in step 230. Such control signal may be associated with the composition of the fragrance product. It may control dosing equipment for dosing of different components of the fragrance product in the production process.

Optionally, if the fragrance product is invalid, e.g. a warning signal for the operator of the production process may be triggered in step 232. Such warning signal may signify the invalidity of the fragrance product. The invalidity may trigger a stop signal for the production process. In such cases, the recipe profile may be updated for the production of the fragrance product to achieve the target temporal evaporation profile.

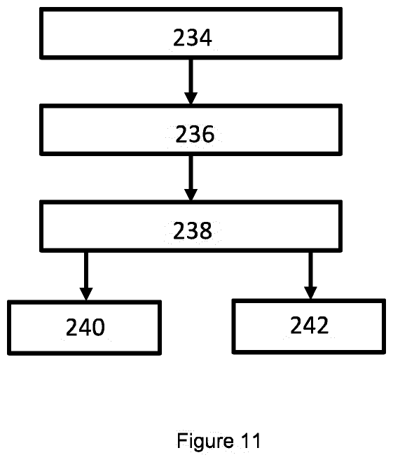

FIG. 11 shows an example of a flowchart for validating the production of the fragrance product.

In step 234, an existing temporal evaporation profile for a fragrance composition is provided, which has been produced from validated precursors.

In step 236, based on the existing temporal evaporation profile a recipe profile is generated according to the method described therein that includes an ingredient identifier and related property data, which are associated with at least one new precursor. New precursors may include a new matrix component or a new fragrance ingredient.

In step 238, the performance characteristic of a fragrance product produced based on the recipe profile and the existing temporal evaporation profile are compared to validate the at least one new precursor. If the comparison lies within an acceptable range, the at least one new precursor is valid. On the other hand, if the comparison does not lie within the acceptable range, the at least one new precursor is invalid.

If new precursor(s) is valid, e.g. control signal is generated for a production process based on the new precursor(s) may be triggered in step 240. Such control signal may by be associated with the composition of the fragrance product including the new precursor. It may control dosing equipment configured to dose different components of the fragrance product in the production process.

If the fragrance product is invalid, e.g. a warning signal for the operator of the production process may be triggered in step 242. Such warning signal may signify the invalidity of the new precursor(s). This may trigger a stop signal for the production process.

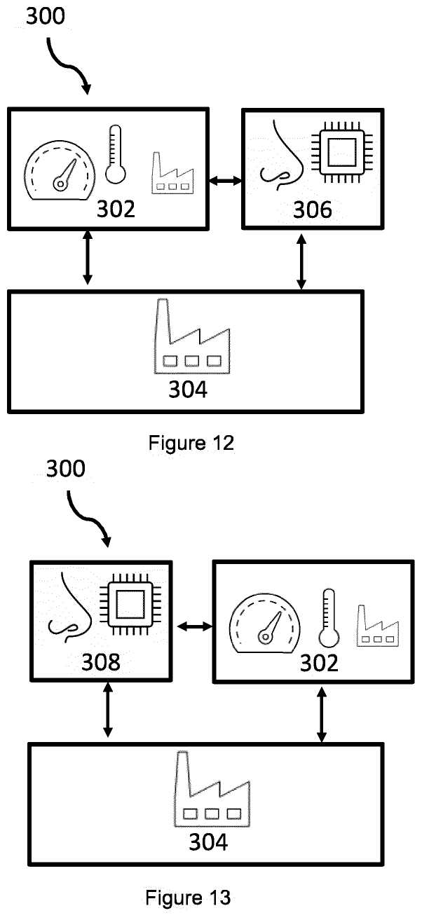

FIG. 12 shows an example of a production line 300 for producing the fragrance product with a monitoring apparatus 306.

The production line 300 may include dosing equipment 302 configured to dose different precursors of the fragrance product in the production process. The production line may include a conveyor system 304 to convey e.g. bottles, plastic packaging or other suitable packaging to be filled with the fragrance product. The production line may include a monitoring apparatus 306 configured to monitor quality of the fragrance product in a production process of the fragrance product.

The monitoring apparatus 306 and/or the dosing equipment apparatus 302 may be configured to receive a target temporal evaporation profile. The target temporal evaporation profile may specify the composition data for the fragrance product including one or more fragrance ingredients and one or more matrix components. The target temporal evaporation profile may include quality criteria like olfactory and/or physio-chemical properties. The monitoring apparatus may be configured to provide the composition data to the dosing equipment and vice versa. The dosing equipment may be configured to control the dosing based on the provided composition data.

The monitoring apparatus 306 may be configured to measure one or more performance characteristic(s). The monitoring apparatus 306 may be configured to compare the olfactory and physio-chemical properties, or any value derived from olfactory and physio-chemical properties to the measured performance characteristic(s). If the comparison lies within an acceptable range or value, the produced fragrant product fulfills quality criteria. If the comparison does not lie within an acceptable range or value, the produced fragrant product does not fulfill quality criteria. In the latter case the monitoring unit may be configured to notify an operator or to provide adjusted composition data to the dosing equipment 302.

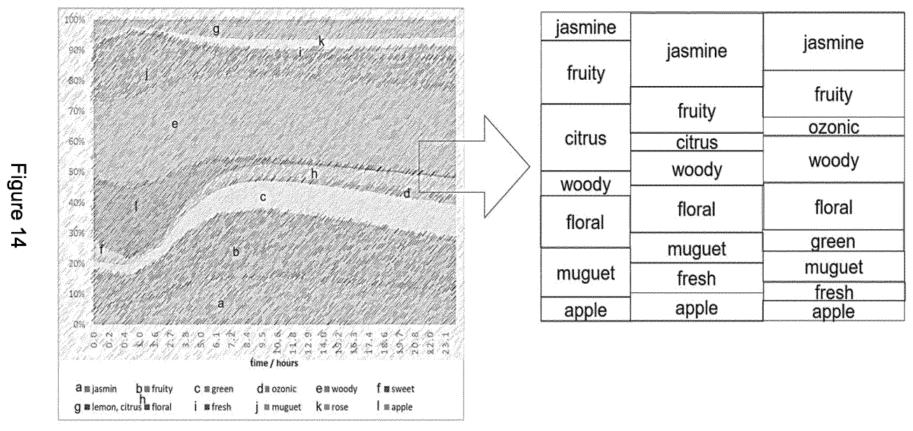

FIG. 13 shows another example of a production line 300 for producing the fragrance product with a validation apparatus 308.

The production line 300 may include dosing equipment 302 configured to dose different precursors of the fragrance product in the production process. The production line 300 may include a conveyor system 304 to convey e.g. bottles, plastic packaging or other suitable packaging to be filled with the fragrance product. The production line 300 may include a validation apparatus 308 configured to validate the production of the fragrance product.

The validation apparatus 308 may be configured to receive an existing temporal evaporation profile. The validation apparatus 308 may be configured to generate a recipe profile based on the existing temporal evaporation profile. The recipe profile may comprise new precursor(s). The validation apparatus 308 may be configured to receive one or more data associated with the new precursor(s). The validation apparatus 308 may be configured to validate the new precursor(s) for production of the fragrance product based on substrate evaporation profile generated for the fragrance product or fragrance composition on a matrix, such as a substrate like skin or wood.

The validation apparatus 308 may be configured to compare the existing and substrate evaporation profile. This way not only the production of the fragrance product but also its application may be validated. The validation apparatus 308 may be configured to provide the composition data including the new precursor(s) to the dosing equipment and vice versa.

Combinations and modifications of the embodiments shown in FIGS. 10 and 11 are similarly possible. Both methods exemplify the strength of the methods described herein. The generation of the temporal evaporation profile of a fragrance product allow for objective assessment of the product in production, since the temporal evaporation profile can be compared to objective performance characteristics of the fragrance product. This allows for simplified and more reliable production through monitoring production of the fragrance product or through validating new precursor(s) to be used for producing the fragrance product.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above. According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

1.-17. (canceled)

18. A computer-implemented method (100) for generating a recipe profile of a fragrance product having a target temporal odour profile, wherein the fragrance product comprises a fragrance composition with one or a plurality of fragrance ingredient(s), comprising the steps of:
a) providing (110) the target temporal odour profile, wherein the target temporal odour profile comprises a time-dependent fractural amount of a plurality of odour families over a predetermined period of time, which is indicative of a desired evaporation behaviour of each odour family in the fragrance composition;
b) providing (120), for each odour family, one or more fragrance ingredients having olfactive contributions matching the respective odour family;
c) selecting (130) at least one ingredient from each odour family to form one or more recipes of the fragrance product, wherein each recipe comprises fragrance composition data associated with the fragrance ingredients of the recipe;
d) determining (140) a temporal odour profile for each recipe;
e) determining (150) a distance of each determined temporal odour profile of the one or more recipes to the target temporal odour profile of the fragrance product;
f) selecting (160) at least one recipe from the one or more recipes that has a distance satisfying a predefined criterion; and
g) providing (170) a recipe profile of the at least one selected recipe preferably usable for production of the fragrance product.

19. The computer-implemented method according to claim 18,
wherein the fragrance product further comprises a matrix; and
wherein each recipe further comprises matrix data associated with the matrix.

20. The computer-implemented method according to claim 18, further comprising:
receiving a measured performance characteristic of the fragrance product produced according to the provided recipe profile, wherein the measured performance characteristic is indicative of a temporal odour profile of the produced fragrance product;
determining a distance between the temporal odour profile of the produced fragrance product and the target temporal odour profile; and
providing, based on the distance, an updated recipe profile of the fragrance product.

21. The computer-implemented method according to claim 18,
wherein steps c)-f) are performed in an iterative process to determine the at least one recipe.

22. The computer-implemented method according to claim 18,
wherein the target temporal odour profile comprises a plurality of evaporation regimes over the predetermined period of time, wherein in each of the plurality of evaporation regimes a fractural amount of odour families are defined.

23. The computer-implemented method according to claim 18,
wherein in step b) the plurality of fragrance ingredients is refined by selecting fragrance ingredients with a particular performance characteristic including a particular physical characteristic, a particular chemical characteristic, or a combination thereof.

24. The computer-implemented method according to claim 18,
wherein in step b) the plurality of fragrance ingredients is provided by including mandatory ingredients and/or excluding undesired ingredients.

25. The computer-implemented method according to claim 18,
wherein step d) further comprises:
d1) receiving (140a) a recipe that comprises fragrance composition data associated with one or more fragrance ingredients of the fragrance composition;
d2) providing (140b) a vapour pressure of each fragrance ingredient based on the fragrance ingredients data;
d3) determining (140c), based on the fragrance ingredients data, a time-dependent interaction coefficient of each fragrance ingredient in a condensed phase of the fragrance composition over a predetermined period of time;
d4) generating (140d), based on the provided vapour pressure and the determined time-dependent interaction coefficient, the temporal evaporation profile of the fragrance product, wherein the temporal evaporation profile is related to a time-dependent quantity associated with the evaporation behaviour of each fragrance ingredient of the fragrance product over the predetermined period of time; and
d5) providing (140e) the generated temporal evaporation profile for the recipe.

26. The computer-implemented method according to claim 25,
wherein the received receipt further comprises matrix data associated with the matrix; and
wherein in step d3), the time-dependent interaction coefficient of each fragrance ingredient in a condensed phase of the fragrance composition in the matrix over a predetermined period of time is determined based on the fragrance ingredients data and the matrix data.

27. The computer-implemented method according to claim 25,
wherein a composition of the gas phase and/or the condensed phase, the interaction coefficient for each fragrance ingredient, the vapour pressure for each fragrance ingredient, or the time-dependent quantity associated with the evaporation behaviour for each fragrance ingredient is determined in a time dependent manner.

28. The computer-implemented method according to claim 18, further comprising:
generating a control file based on the recipe profile of the at least one selected recipe, which is usable for controlling production of the fragrance product.

29. A method for monitoring production of a fragrance product, the method comprising the steps of:
providing (220) a target temporal evaporation profile;
providing (222) a performance characteristic of a produced fragrance product that has a recipe profile generated according to the method of claim 18; and
comparing (224) the performance characteristic with the target temporal evaporation profile to determine if the produced fragrance product fulfils predetermined quality criteria.

30. A method for validating production of a fragrance product, the method comprising the steps of:
providing (234) an existing temporal evaporation profile for a fragrance composition that has been produced from validated precursors;
generating (236) a recipe profile based on the existing temporal evaporation profile according to the method of claim 18, wherein the recipe profile comprises an ingredient identifier and related property data, which are associated with at least one new precursor; and
comparing a performance characteristic of a fragrance product produced using the recipe profile and the existing temporal evaporation profile to validate the at least one new precursor.

31. An apparatus for generating a recipe profile of a fragrance product having a target temporal odour profile, wherein the fragrance product comprises a fragrance composition with one or a plurality of fragrance ingredient(s) and a matrix, the apparatus comprising one or more processing unit(s) configured to generate the recipe profile of the fragrance product, wherein the processing unit(s) include instructions, which when executed on the one or more processing unit(s) execute the method steps of claim 18.

32. An apparatus for monitoring production of a fragrance product, the apparatus comprising one or more processing unit(s) configured to monitor production, wherein the processing unit(s) include instructions, which when executed on the one or more processing unit(s) execute the method steps of claim 29.

33. A computer program element comprising instructions, which when executed by a processing unit, cause the processing unit to carry out the steps of the method of claim 18.

34. Use of recipe profile generated in a method according to claim 18 for quality control and/or verification purposes.
